(19) United States
(12) Patent Application Publication
(10) Pub. No.: US 2005/0201970 A1
(43) Pub. Date: Sep. 15, 2005

(54) Bed sore medicine cream

(76) Inventor: Mary D. Hu, El Monte, CA (US)

Correspondence Address: RAYMOND Y. CHAN, 108 N. YNEZ AVE., SUITE 128, MONTEREY PARK, CA, 91754, US

(21) Appl. No.: 10/959,630

(22) Filed: Oct. 5, 2004

Related U.S. Application Data
(60) Provisional application No. 60/552,200, filed on Mar. 12, 2004.

(51) Int. Cl. A61K 35/78; A61K 7/06

(52) U.S. Cl. 424/74; 424/748; 424/778

(57) ABSTRACT

A skin condition relieving medication, which can be directly applied onto a skin condition prone area of a user, is provided for preventing infection and reducing inflammation and swelling, while allowing air to reach the skin condition prone area in order to dry the skin condition and eventually healing the skin condition, wherein the skin condition relieving medication includes a predetermined amount of primary herb mixture including one or more herbs chosen from forsythia, frankincense, myrrh, cuttlefish bone, honeysuckle, turmeric and garden angelica, provided for the treating of the skin conditions, a predetermined amount of catalyst including vitamin E, and a predetermined amount of binding agent, which most suitably is Vaseline containing the primary herb mixture and the catalyst, and binding the primary herb mixture to the skin condition prone area of the user.

### CROSS-REFERENCE OF RELATED APPLICATION

This is a regular application of a provisional application having an application No. 60/552,200 and a filing date of Mar. 12, 2004.

### BACKGROUND OF THE PRESENT INVENTION

1. Field of Invention

This version of the invention is concerned with the field of medicated creams, ointments, and salve for wounds, bed sores, ulcers, and other similar afflictions. More specifically, this version of the invention is concerned with a topical cream, ointment, or salve having medicinal properties that promote healing of a bed sore, wound, ulcer, or other affliction.

2. Description of Related Arts

People or hospital patients who are bedridden for extended periods of time become afflicted with various skin maladies, such as wounds, sores, ulcers, and various surface conditions, as a result of poor blood and air circulation. The most common form of affliction is known as a decubitus ulcer or bed sore. The part of the body making contact with a bed or support device is under pressure by the weight of the body urging against the bed or support device, thus resulting in muscle and associated tissue and becoming compressed and stiffened. This situation is most acute when the bone of a patient presses on the tissue, such as the spine, coccyx or tailbone, hips, heels, and elbow. The weight of the patient's body presses upon the bone, which presses upon adjacent tissue and skin. As a result, blood circulation decreases and the tissue beings to decay. The patent may also experience muscle pain and cramps as normal levels of oxygen and nutrition that would otherwise be delivered to muscles are decreased because of reduced blood circulation. The outer layer of the skin or epidermis becomes weakened and loses its resiliency and ability to sustain injury and heal.

Furthermore, the part of the body making direct contact with the bed or support surface is prevented from receiving an adequate supply or air or air circulation necessary to promote healing of any wounds, ulcers, or sores caused by an extended stay in a bed.

Ulcers or bed sore are generally characterized in severity over five stages: Stage 1, characterized by surface discoloration (pinkish or reddening) of the kin; Stage 2, broken or unbroken blistering; Stage 3, extension of wound through all layers of the skin; Stage 4, extension of wound through the skin, underlying muscle, tendon, and bone; and Stage 5, generally referring to an extremely deep wound affecting underlying organs and bone.

The most common method of prevention involves having the patient change position over a regular interval, generally every two hours, so as to relieve and distribute pressure and weight over the body. In some cases, the patient can change position unaided, but in many situations nursing care must be provided to help a patient change position at the prescribed interval. The changing of position typically involves rolling the patient, although other methods and procedures may be used depending upon the condition of the patient.

However, not all patients can change position at regular intervals to relieve pressure so as to prevent the formation of an ulcer or to prevent the worsening of an existing ulcer. In such situations, a cushion or other device may be positioned under or adjacent to the wound between the patient and the bed to relieve pressure or redistribute pressure away from the wound. Nevertheless, bed sores may still develop even with use of such cushions or devices, and topical treatment of bed sores then becomes necessary.

A common method of treating bed sores is to cover the sores with a patch or dressings, while accomplishing their intended function, have their own drawbacks and disadvantages. For instance, patches and dressings completely cover a bed sore and thus prevent ambient air from reaching the bed sore, which would otherwise permit the bed sore the dry out and eventually heal. The bed sore thus remains as an open wound because of the lack of air, and healing is retarded indefinitely.

What is needed then to overcome the aforementioned disadvantages of patches and dressings for bed sores is the provision of a bed sore medicine cream that can be applied to a bed sore so as protect the bed sore from infection and to reduce inflammation and swelling, while allowing ambient air to reach the bed sore in order to promote eventual healing of the bed sore. The bed sore medicine cream that is the subject of the instant invention uses a variety of Chinese herbs having properties that control and heal bed sores, such as anti-biotic and anti-inflammatory ingredients, a catalyst to activate medicinal ingredients of two or more Chinese herbs, and a carrier or binding agent to contain the herbs and catalyst and maintain said herbs and catalyst against the bed sore until healing of said bed sore occurs.

Numerous designs for treatments and creams have been provided in the prior art for topical treatment of bed sores, wounds, ulcers, and the like. Even though these treatments may be suitable for the specific individual purposes to which they address, they would not be suitable for the purposes of the present version of the invention. Such designs are exemplified by:

U.S. Pat. No. 4,719,111, Novel Composition for Treatment of Decubitus Ulcers, Issued to Wilson on 12 Jan. 1988;
U.S. Pat. No. 5,350,774, Therapeutic Preparation for Topical Application to the Skin, Issued to Palou on 27 Sep. 1994;
U.S. Pat. No. 5,494,668, Method of Treatment Musculoskeletal Disease and a Novel Composition Therefor, Issued to Patwardhan on 27 Feb. 1996;
U.S. Pat. No. 5,804,206, Therapeutic Composition and Method for Treating Skin Using Centipeda Cunningham Extract, Issued to D'Amelio et al on 8 Sep. 1998;
U.S. Pat. No. 6,277,396, Dietary Supplement Containing a Thermogenic Substance and an Adrenal Support Substance, Issued to Dente on 21 Aug. 2001; and

U.S. Pat. No. 6,534,086, Composition and Method for Treatment of Inflammation and Pain in Mammals, Issued to Krumhar on 18 Mar. 2003.

As illustrated by the background art, efforts are continuously being made in an attempt to develop devices for topical treatments for wounds, bed sores, ulcers, and the like. No prior effort, however, provides the benefits attendant with the present invention in which two or more Chinese herbs are combined in equal or varying quantities along with a catalyst and carrier or binding agent so as to produce a bed sore medicine cream. The herbs within the bed sore medicine cream contain anti-biotic and anti-inflammatory agents, which promote healing of the bed sore. Furthermore, the bed sore medicine cream allows air to reach the bed sore, which ultimately promotes drying and eventual healing of the bed sore.

As such, it may be appreciated that there is a continuing need for a new and improved bed sore medicine. In these respects, the present version of the invention substantially departs from the conventional concepts and designs of the prior art, and in so doing provides an apparatus that substantially fulfills this need. Additionally, the prior patents and commercial techniques do not suggest the present inventive combination of component elements and ingredients arranged and configured as disclosed herein.

The present invention achieves its intended purposes, objects, and advantages through a new, useful and unobvious combination of method steps and components elements, with the u se of a minimum number of functioning ingredients, at a reasonable cost to acquire and assemble, and by employing only readily available materials.

### SUMMARY OF THE PRESENT INVENTION

The present version of the invention, which will be described in greater detail hereinafter, relates to the field of medicated creams, ointments, and salves for wounds, bed sores, ulcers, and other similar afflictions. More specifically, this version of the invention is concerned with a topical cream, ointment, or salve having medicinal properties that promote healing of a bed sore, wound, ulcer, or other affliction. My version of the invention overcomes all of the shortcomings listed previously, in addition to novel aspects that will be described in detail hereinafter.

The bed sore medicine cream is a topical treatment that is to applied directly to an open bed sore, ulcer, or the like in order to promote healing thereof. The bed sore medicine cream is comprised of at least two different primary Chinese herbs containing anti-biotic and anti-inflammatory agents so as to prevent infection and swelling, a catalyst that activates the medicinal and healing capabilities of the herbs and a carrier and binding agent that holds the herbs and catalyst together and against the bed sore and adjacent tissue. Furthermore, the carrier and binding agent allow eventual passage of air therethrough so that said air can reach the bed sore located under the cream so as to promote drying and healing of the bed sore. The bed sore medicine cream can be enhanced by addition of one or more secondary Chinese herbs to the primary Chinese herbs.

Described briefly, according to the first embodiment, the bed sore medicine cream is comprised of equal or varying parts of Chinese herbs Lian Qiao, Ru Xiang, and Mo Yao, dried and processed to a fine or granular power. The herbs are added to a quantity of Vitamin E and Vaseline, said Vaseline functioning as a binding or carrier agent.

In a second embodiment, the bed sore medicine cream is comprised of equal or varying parts of Chinese herbs Lian Qiao, Ru Xiang, Mo Yao, and Cuttlefish bone Hai Piao Xiao or Wu Zei Gu, dried and processed to a fine or granular power. The herbs and bone are added to a quantity of Vitamin E and Vaseline, said Vaseline Functioning as a binding or carrier agent.

In a third embodiment, the bed sore medicine cream is comprised of equal or varying parts of Chinese herbs Ru Xiang and Mo Yao, dried and processed to a fine or granular power. The herbs are added to a quantity of Vitamin E and Vaseline, said Vaseline functioning as a binding or carrier agent.

In a fourth embodiment, the bed sore medicine cream is comprised of equal or varying parts of Chinese herbs Lian Qiao, Jin Yin Hua, Mo Yao, and Jiang Huang, dried and processed to a fine or granular power. The herbs are added to a quantity of Vitamin E and Vaseline, said Vaseline functioning as a binding or carrier agent.

In a fifth embodiment, the bed sore medicine cream is comprised of equal or varying parts of Chinese herbs Lian Qiao, Jin Yin Hua, Mo Yao, Jiang Huang, and Bai Zhi, dried and processed to a fine or granular power. The herbs are added to a quantity of Vitamin E and Vaseline, said Vaseline functioning as a binding or carrier agent.

The bed sore medicine cream is applied directly over and to the bed sore, ulcer, or the like. The anti-biotic and anti-inflammatory ingredients of the herbs bone function reduce swelling and prevent infection of the bed sore. The carrier or binding agent of the cream permits passage of ambient air therethrough so that air eventually makes contact with the bed sore under the cream and tissue adjacent to the cream so as to promote drying and eventual healing of the bed sore.

Furthermore, the aforementioned embodiments of the bed sore medicine cream can be modified by adding one or more secondary Chinese herbs, adjusting either the number of herbs and particular quantities thereof to the four embodiments of the bed sore cream described previously. These additional herbs include common Chinese herbs, such as Da Qing Ye, Ban Lan Gen, Qing Dai, Pu Gong Ying, Zi Hua Di Ding, Yu Xing Cao, Bai Jiang Cao, Ma Chi Xian, Tu Fu Ling, Bai Xian Pi, She Gan, San Dou Gen, Chuan Xin Lian, Dan Gui, Hu Zhang, Jiang Xiang, Bai Tou Weng, and the like.

My invention, therefore, resides not in any one of these features per se, but rather in the particular combination of all of them herein disclosed. It is distinguished from the prior art in this particular combination of all of its components and ingredients for the functions specified.

In order that the detailed description of the invention may be better understood and that the present contribution to the art can be more fully appreciated, additional features of the invention will be described hereinafter. It should be appreciated by those skilled in the art that the conception and the disclosed specific methods and structures may be readily utilized as a basis for modifying or designing other structures for carrying out the same purposes of the present invention. It should be realized by those skilled in the art that such equivalent methods and ingredients do not depart from the spirit and scope of the invention.

In this respect, before explaining at least one embodiment of the invention in detail, it is to be understood that the invention is not limited in its application to the details of ingredients and components and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other mixtures, creams, methods and systems for treating bed sores and carrying out the several purposes of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application nor is it intended to be limited as to the scope of the invention in anyway.

Accordingly, it is an object of my version of the invention to provide a low-cost, easy-to-manufacture, and easy-to-market bed sore medicine cream.

A further object of my version of the invention is to provide an easy-to-use, versatile, and adaptable bed sore medicine cream.

A significant object of the invention is to provide a bed sore medicine cream in various embodiments comprised of a mixture of two or more primary Chinese Herbs, a catalyst and a binding or carrier agent, said primary herbs comprised in equal or varying parts of Lian Qiao, Ru Xiang, Mo Yao, Cuttlefish bone Wu Zei Gui, Jin Yin Hua, Jiang Huang, and Bai Zhi, and a variety of secondary Chinese herbs.

A final but very significant object of the invention is to provide a bed sore medicine cream that can be applied to a bed sore, wound, ulcer, and the like so as to prevent infection and to reduce inflammation and swelling, while allowing ambient air to reach the bed sore in order to dry the bed sore and promote eventual healing thereof.

For a better understanding of the invention, its operating advantages and the specific objects attained by its uses, reference should be made to the accompanying drawings and descriptive matter in which there are illustrated preferred embodiments of the invention. The foregoing has outlined some of the more pertinent objects of the invention. These objects should be construed to be merely illustrative of some of the more prominent features and applications of the present invention. Many other beneficial results can be attained by applying the disclosed invention in a different manner or by modifying the invention within the scope of the disclosure. Accordingly, other objects and a fuller understanding of the invention may be had by referring to the summary of the invention and the detailed description of the preferred embodiments in addition to the scope of the invention illustrated by the accompanying drawings.

Accordingly, in order to accomplish the above objects, the present invention provides a skin condition relieving medication directly applicable on a skin condition prone area of a user, wherein the skin condition relieving medication treats skin conditions on the skin condition prone area, wherein the medication comprises:
a predetermined amount of primary herb mixture, comprising a predetermined amount of Forsythia, a predetermined amount of Frankincense, and a predetermined amount of additional herb mixture, wherein the Forsythia has a Chinese herb name of Lian Qiao which has forsythin, matairesinoside, betulinic acid, phyillygenin, and pinoresinol, wherein the Forsythia is provided for lowering fever, promoting anti-inflammation, protecting the liver, ceasing vomiting and promoting blood circulation and urination, and the Frankincense has a Chinese herb name of Ru Xiang, provided for reducing pain, swelling and inflammation, wherein the additional herb mixture is mixed with the Forsythia and the Frankincense to enhance the treating of the skin conditions;
a predetermined amount of catalyst comprising vitamin E; and
a predetermined amount of a binding agent, provided for containing the primary herb mixture and the catalyst, and binding the primary herb mixture to a tissue layer of the skin condition prone area of the user.

### BRIEF DESCRIPTION OF THE DRAWINGS

The foregoing and other objects, features and advantages of the invention will become more fully understood from the following description of the preferred embodiments of the invention as illustrated in the accompanying drawings. The drawings are not necessarily to scale, emphasis instead being placed upon illustrating the components of the invention.

FIG. 1 is an illustration of herb in natural form, known in Chinese as Lian Qiao.

FIG. 2 is an illustration of herb in natural form, known in Chinese as Ru Xiang.

FIG. 3 is an illustration of herb in natural form, known in Chinese as Mo Yao.

FIG. 4 is an illustration of herb in natural form, known in Chinese as Wu Zei Gui.

FIG. 5 is an illustration of herb in natural form, known in Chinese as Jin Yin Hua.

FIG. 6 is an illustration of herb in natural form, known in Chinese as Jiang Huang.

FIG. 7 is an illustration of herb in natural form, known in Chinese as Bai Zhi.

### DETAILED DESCRIPTION OF THE PREFERRED EMBODIMENT

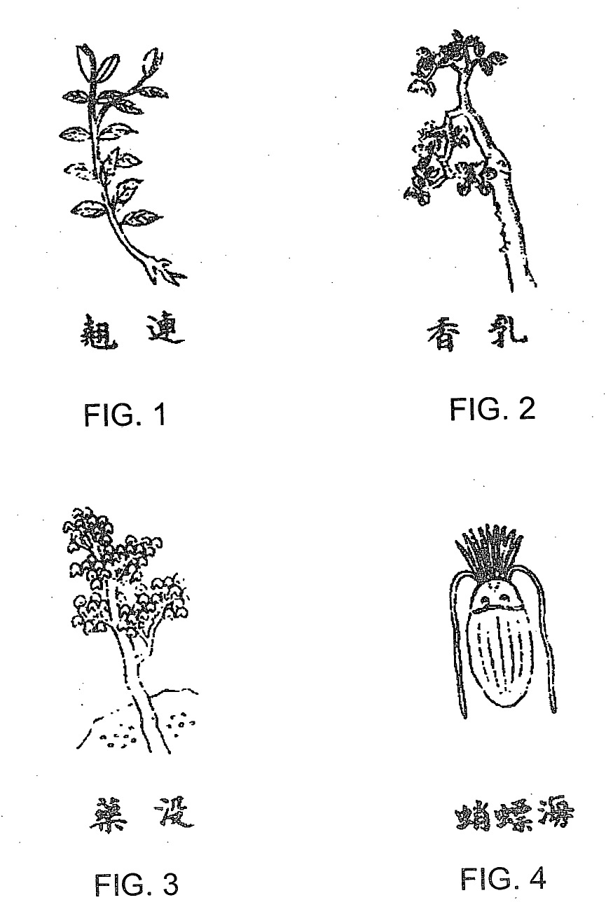

Referring now to the drawings and, in particular, to FIG. 1 to FIG. 7 wherein there are illustrated various medicinal and therapeutic herbs in their natural form. The herbs function as the primary herbs within this version of the invention and can be dried and processed into a powdery or granular texture and combined with two or more of each other granular texture and combined with two or more of each other in specific quantities to achieve a particular medicinal, therapeutic effect. A binding agent and carrier are added to the combination of two or more hers to facilitate application and efficacy of the primary herbs. The bed sore medicine cream created with the primary herbs can be enhanced with the addition of one or more secondary Chinese herbs in varying quantities, as will be referenced later in this description.

Referring to FIG. 1, therein illustrated in natural form is the herb known in Chinese as Lian Qiao, commonly known in Western taxonomy as Forsythia. The herb provides a broad spectrum antibiotic effect and can be used to lower fever, promote anti-inflammation, protect the liver, stop vomiting, and promote blood circulation and urination. Its chemical ingredients include forsythin, matairesinoside, betulinic acid, phyillygenin, and pinoresinol.

In FIG. 2, therein is illustrated in natural form the herb know in Chinese as Ru Xiang, commonly known in Western taxonomy as Frankincense or Mastic. The herb reduced pain, swelling, and inflammation. It is especially effective as an anti-inflammatory agent in the treatment of boils, psoriasis, and other afflictions of the epidermis. Chinese Boswellia carterii is sometimes called mastic and not to be confused with Pistacia lentiscus, also called mastic, which is used to treat ulcers.

Therein illustrated in FIG. 3 in natural form is the herb known in Chinese as Mo Yao, commonly known in Western taxonomy as Myrrh. The herb functions as an anti-inflammatory and anti-bacterial agent, reducing pain and swelling and also potent enough to soften swellings and carbuncles.

Referring to FIG. 4, therein illustrated in natural form and referred to in Chinese as Hai Piao Xiao or Wu Zei Gu, commonly known in Western taxonomy as Cuttlefish bone. The bone is known to have properties that stop bleeding, restore kidney deficiencies, and promote healing of rashes and ulcers.

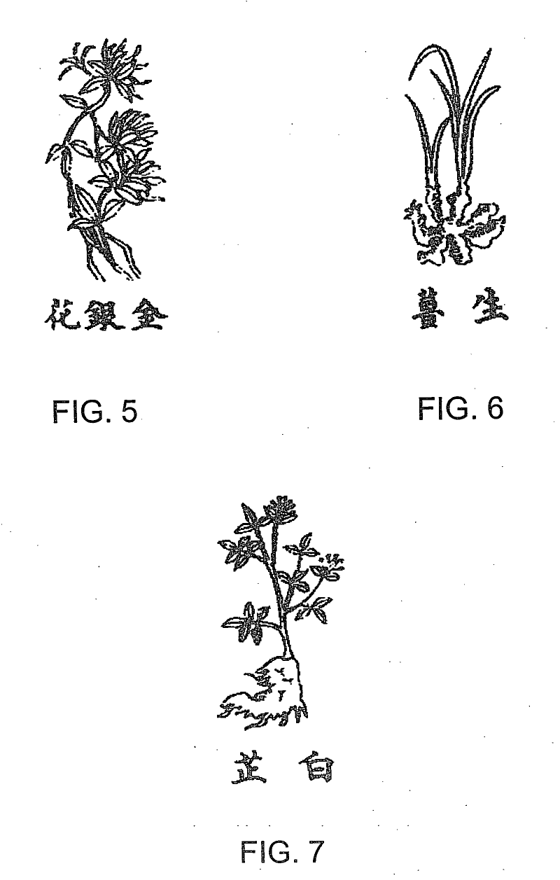

In FIG. 5, therein illustrated in natural form the herb known in Chinese as Jin Hin Hua, commonly known in Western taxonomy as Honeysuckle. Flowers, leaves, and bark are used. The herb is used primarily to treat “hot” infectious disorders, such as abscesses, sores, and inflammation.

Therein illustrated in FIG. 6 in natural form is the herb known in Chinese as Jiang Huang, commonly known in Western taxonomy as Turmeric. Turmeric is the dried rhizome of Aromati Turmeric of the Ginger family. The active ingredients of turmeric are its essence oil, fat, curcumin, and the like. The root and rhizome, the underground stem, are used medicinally. The active ingredient, curcumin, reduces inflammation by lowering histamine levels and possibly by increasing production of natural cortisone by the adrenal glands.

In FIG. 7, therein illustrated in natural form the herb known in Chinese as Bai Zhi, commonly known in Western taxonomy as Garden Angelica, Wild Angelica, and Root of the Holy Ghost. The herb alleviates pain, clears and reduces swelling and inflammation, expels pus from carbuncles, sores, and ulcers, and expels dampness. It can be used topically as a multi-ingredient preparation. The part of the plant providing the medicinal and therapeutic effect is the root.

The bed sore medicine cream that is the subject of this version of the invention is provided in four embodiments or compounds, comprised in equal (or varying) parts or quantities of said primary herbs and a catalyst and binding or carrier agent, such as a cream or gel, which functions to contain the mixture of herbs and catalyst and bind to the bed sore and surrounding tissue layer, thereby ensuring that the mixture of herbs and catalyst make contact with the afflicted area and provide a healing effect. The carrier or binding agent also allows ambient air to reach the bed sore so as to promote drying and eventual healing of the bed sore.

In a first embodiment of this version of the invention, a medicinal bed sore cream is comprised of equal parts of Lian Qiao, Ru Xiang, and Mo Yao, all provided in similar form, i.e. dried and processed to a fine or granular power. Vitamin E is added to function as a catalyst along with Vaseline, which performs as a binding or carrier agent.

In a second embodiment of this version of the invention, a medicinal bed sore cream is comprised of equal parts of Lian Qiao, Ru Xiang, Mo Yao, and Hai Piao Xiao or Wu Zei Gu, all provided in similar form, i.e. dried and processed to a fine or granular power, Vitamin E is added as a catalyst along with Vaseline, which performs as a binding or carrier agent.

In a third embodiment of the invention, a medicinal bed sore cream is comprised of equal parts of Ru Xiang and Mo Yao, all provided in similar form, i.e. dried and processed to a fine or granular power. Vitamin E is added as a catalyst along with Vaseline, which performs as a binding or carrier agent.

In a fourth embodiment of this version of the invention, a medicinal bed sore cream is comprised of equal parts of Lian Qiao, Jin Yin Hua, Mo Yao, and Jiang Huang, all provided in similar form, i.e. dried and processed to a fine or granular power. Vitamin E is added as a catalyst along with Vaseline, which performs as a binding or carrier agent.

In a fifth embodiment of this version of the invention, a medicinal bed sore cream is comprised of equal parts of Lian Qiao, Jin Yin Hua, Mo Yao, Jiang Huang, and Bai Zhi, all provided in similar form, i.e. dried and processed to a fine or granular power. Vitamin E is added as a catalyst along with Vaseline, which performs as a binding or carrier agent.

A quantity of the bed sore medicine cream in any of the aforementioned embodiments is applied directly over and to the bed sore, wound, or the like. The medicinal and therapeutic properties of the cream will promote healing of the bed sore and obviate the need for closed dressings or patches. The bed sore medicine cream can be re-applied as needed to ensure that healing of the bed sore continues unabated. If necessary, the quantity of any particular herb in relation to quantities of other herbs within a particular embodiment or compound can be varied to customize or tailor the medicinal affects of a particular embodiment of the bed sore medicine cream. For instance, increasing or decreasing the quantity of a particular herb within a particular embodiment of the bed sore medicine cream will likewise increase or decrease its medicinal and therapeutic function within said embodiment of the bed sore cream.

Similarly, the aforementioned embodiments of the bed sore medicine cream can be enhanced by adding one or more of the following secondary herbs, adjusting either the number of herbs and particular quantities thereof to the four embodiments or the bed sore cream described previously. These additional herbs include, but are not limited to the following:
Da Qing Ye (Isatis leaf)
Ban Lan Gen (Isatis root)
Qing Dai (Natural Indigo)
Pu Gong Ying (Dandelion)
Zi Hua Di Ding (Viola)
Yu Xing Cao (Houttuynia)
Bai Jiang Cao (Thlaspi)
Ma Chi Xian (Portulaca)
Tu Fu Ling (Smilax)
Bai Xian Pi (Dictamnus)
She Gan (Belamcanda, Blackberry Lily)
San Dou Gen (Sophora Root)
Chuang Xin Lian (Green Chiretta)
Dang Gui (Angelica Root)
Hu Zhang (Japanese Knottweed)
Jiang Xiang (Dalbergia Heartwood)
Bai Tou Weng (Chinese Anemone Root)

While this version of the invention has been illustrated and described in detail in the drawings and foregoing description, the same is to be considered as illustrative and not restrictive in character, it being understood that only the preferred embodiments have been shown and described and that all changes and modifications that come within the spirit of the version of the invention are desired to be protected. With respect to the above description then, it is to be realized that the optimum relationships for the ingredients of the invention, to include variations in quantity, texture, function and use, are deemed readily apparent and obvious to one skilled in the art, and all equivalent relationships to those illustrated in the drawings and described in the specification are intended to be encompassed by the present invention.

From the foregoing, it will be understood by persons skilled in the art that an improved bed sore medicine cream has been provided. The invention is relatively simple and easy to create, yet affords a variety of uses. While my description contains many specificities, these should not be construed as limitations on the scope of the version of the invention, but rather as an exemplification of the preferred embodiment thereof. The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be resorted to, falling within the scope of the invention. Although this invention has been described in its preferred form with a certain degree of particularity, it is understood that the present disclosure of the preferred forms have been made only by way of example and numerous changes in the details of construction and combination and arrangement of components and ingredients may be resorted to without departing from the spirit and scope of the invention.

1. A skin condition relieving medication directly applicable on a skin condition prone area of a user, wherein said skin condition relieving medication treats skin conditions on said skin condition prone area, wherein said medication comprises:
a predetermined amount of primary herb mixture, comprising a predetermined amount of Forsythia, a predetermined amount of Frankincense, and a predetermined amount of additional herb mixture, wherein said Forsythia has a Chinese herb name of Lian Qiao which has forsythin, matairesinoside, betulinic acid, phyillygenin, and pinoresinol, wherein said Forsythia is provided for lowering fever, promoting anti-inflammation, protecting the liver, ceasing vomiting and promoting blood circulation and urination, and said Frankincense has a Chinese herb name of Ru Xiang, provided for reducing pain, swelling and inflammation, wherein said additional herb mixture is mixed with said Forsythia and said Frankincense to enhance the treating of said skin conditions;
a predetermined amount of catalyst comprising vitamin E; and
a predetermined amount of a binding agent, provided for containing said primary herb mixture and said catalyst, and binding said primary herb mixture to a tissue layer of said skin condition prone area of said user.

2. The skin condition relieving medication, as recited in claim 1, wherein said medication comprises 40% to 50% by volume of said binding agent, 5% to 10% by volume of said catalyst and 40% to 50% by volume of said primary herb mixture.

3. The skin condition relieving medication, as recited in claim 2, wherein said binding agent is a gel.

4. The skin condition relieving medication, as recited in claim 2, wherein said binding agent is a cream.

5. The skin condition relieving medication, as recited in claim 4, wherein said additional herb mixture comprises a predetermined amount of Myrrh, having a Chinese herb name of Mo Yao, provided for promoting anti-inflammatory and anti-bacterial, reducing pain and swelling, and softening swellings and carbuncles.

6. The skin condition relieving medication, as recited in claim 5, wherein said primary herb mixture comprises 33% by volume of said Forsythia, 33% volume of said Frankincense and 33% by volume of said Myrrh.

7. The skin condition relieving medication, as recited in claim 5, wherein said additional herb mixture further comprises a predetermined amount of Cuttlefish Bone having a Chinese herb name of Hai Piao Xiao, provided for ceasing bleeding, restoring kidney deficiencies, and promoting healing of rashes and ulcers.

8. The skin condition relieving medication, as recited in claim 7, wherein said primary herb mixture comprises 25% by volume of said Forsythia, 25% by volume of said Frankincense, 25% by volume of said Myrrh and 25% by volume of said Cuttlefish Bone.

9. The skin condition relieving medication, as recited in claim 4, wherein said additional herb mixture comprises a predetermined amount of Honeysuckle having a Chinese herb name of Jin Yin Hua, and a predetermined amount of Turmeric having a Chinese herb name of Jiang Huang, wherein said Honeysuckle is provided for treating abscesses, sores and inflammation, and said Turmeric is provided for reducing inflammation by lowering histamine levels and increasing a production of natural cortisone by adrenal glands of said user.

10. The skin condition relieving medication, as recited in claim 9, wherein said primary herb mixture comprises 25% by volume of said Forsythia, 25% by volume of said Frankincense, 25% by volume of said Honeysuckle and 25% by volume of said Turmeric.

11. The skin condition relieving medication, as recited in claim 9, wherein said additional herb mixture further comprises a predetermined amount of Garden Angelica, having a Chinese herb name of Bai Zhi, provided for alleviating pain, clearing and reduces swelling and inflammation, expelling pus from carbuncles, sores and ulers, and expelling dampness.

12. The skin condition relieving medication, as recited in claim 11, where said primary herb mixture comprises 20% by volume of said Forsythia, 20% by volume of said Frankincense, 20% by volume of said Honeysuckle, 20% by volume of said Turmeric and 20% by volume of said Garden Angelica.

13. The skin condition relieving medication, as recited in claim 4, wherein said cream is Vaseline.

14. The skin condition relieving medication, as recited in claim 1, wherein said medication further comprises one or more secondary herb, chosen from a secondary herb group, having isatis leaf, isatis root, natural indigo, dandelion, viola, houttuynia, thlaspi, partulaca, smilax, dictamnus, belamcanda, sophora root, green chiretta, angelica root, Japaneses knottweed and dalbergia heartwood.

15. A skin condition relieving medication directly applicable on a skin condition prone area of a user, wherein said skin condition relieving medication treats skin conditions on said skin condition prone area, wherein said medication comprises:
a predetermined amount of primary herb mixture, comprising a predetermined amount of Myrrh, and a predetermined amount of Frankincense, wherein said Myrrh has a Chinese herb name of Mo Yao, provided for promoting anti-inflammatory and anti-bacterial, reducing pain and swelling, and softening swellings and carbuncles, and said Frankincense has a Chinese herb name of Ru Xiang, provided for reducing pain, swelling and inflammation, wherein said primary herb mixture is provided for treating said skin conditions;
a predetermined amount of catalyst comprising vitamin E; and
a predetermined amount of a binding agent, provided for containing said primary herb mixture and said catalyst, and binding said primary herb mixture to a tissue layer of said skin condition prone area of said user.

16. The skin condition relieving medication, as recited in claim 15, wherein said medication comprises 40% to 50% by volume of said binding agent, 5% to 10% by volume of said catalyst and 40% to 50% by volume of said primary herb mixture.

17. The skin condition relieving medication, as recited in claim 16, wherein said binding agent is a gel.

18. The skin condition relieving medication, as recited in claim 16, wherein said binding agent is a cream.

19. The skin condition relieving medication, as recited in claim 18, wherein said primary herb mixture comprises 50% by volume of said Myrrh and 50% by volume of said Frankincense.

20. The skin condition relieving medication, as recited in claim 18, wherein said cream is Vaseline.

21. The skin condition relieving medication, as recited in claim 15, wherein said medication further comprises one or more secondary herb, chosen from a secondary herb group, having isatis leaf, isatis root, natural indigo, dandelion, viola, houttuynia, thlaspi, partulaca, smilax, dictamnus, belamcanda, sophora root, green chiretta, angelica root, Japaneses knottweed and dalbergia heartwood.

22. A skin condition relieving medication directly applicable on a skin condition prone area of a user, wherein said skin condition relieving medication treats skin conditions on said skin condition prone area, wherein said medication comprises:
a predetermined amount of primary herb mixture, comprising a predetermined amount of Forsythia, a predetermined amount of Honeysuckle, and a predetermined amount of additional herb mixture, wherein said Forsythia has a Chinese herb name of Lian Qiao which has forsythin, matairesinoside, betulinic acid, phyillygenin, and pinoresinol, wherein said Forsythia is provided for lowering fever, promoting anti-inflammation, protecting the liver, ceasing vomiting and promoting blood circulation and urination, and said Honeysuckle has a Chinese herb name of Jin Yin Hua, provided for treating abscesses, sores and inflammation, wherein said additional herb mixture is mixed with said Forsythia and said Honeysuckle to enhance the treating of said skin conditions;
a predetermined amount of catalyst comprising vitamin E; and
a predetermined amount of a binding agent, provided for containing said primary herb mixture and said catalyst, and binding said primary herb mixture to a tissue layer of said skin condition prone area of said user.

23. The skin condition relieving medication, as recited in claim 22, wherein said medication comprises 40% to 50% by volume of said binding agent, 5% to 10% by volume of said catalyst and 40% to 50% by volume of said primary herb mixture.

24. The skin condition relieving medication, as recited in claim 23, wherein said binding agent is a gel.

25. The skin condition relieving medication, as recited in claim 23, wherein said binding agent is a cream.

26. The skin condition relieving medication, as recited in claim 25, wherein said additional herb mixture comprises a predetermined amount of Myrrh, having a Chinese herb name of Mo Yao, provided for promoting anti-inflammatory and anti-bacterial, reducing pain and swelling, and softening swellings and carbuncles.

27. The skin condition relieving medication, as recited in claim 26, wherein said primary herb mixture comprises 33% by volume of said Forsythia, 33% volume of said Honeysuckle and 33% by volume of said Myrrh.

28. The skin condition relieving medication, as recited in claim 25, wherein said additional herb mixture comprises a predetermined amount of Garden Angelica, having a Chinese herb name of Bai Zhi, provided for alleviating pain, clearing and reduces swelling and inflammation, expelling pus from carbuncles, sores and ulers, and expelling dampness.

29. The skin condition relieving medication, as recited in claim 28, wherein said primary herb mixture comprises 33% by volume of said Forsythia, 33% volume of said Honeysuckle and 33% by volume of said Garden Angelica.

30. The skin condition relieving medication, as recited in claim 26, wherein said additional herb mixture further comprises a predetermined amount of Garden Angelica, having a Chinese herb name of Bai Zhi, provided for alleviating pain, clearing and reduces swelling and inflammation, expelling pus from carbuncles, sores and ulers, and expelling dampness.

31. The skin condition relieving medication, as recited in claim 30, wherein said primary herb mixture comprises 25% by volume of said Forsythia, 25% volume of said Honeysuckle, 25% by volume of Myrrh and 25% by volume of said Garden Angelica.

32. The skin condition relieving medication, as recited in claim 28, wherein said additional herb mixture further comprises a predetermined amount of Cuttlefish Bone having a Chinese herb name of Hai Piao Xiao, provided for ceasing bleeding, restoring kidney deficiencies, and promoting healing of rashes and ulcers.

33. The skin condition relieving medication, as recited in claim 32, wherein said primary herb mixture comprises 25% by volume of said Forsythia, 25% volume of said Honeysuckle, 25% by volume of Garden Angelica and 25% by volume of said Cuttlefish Bone.

34. The skin condition relieving medication, as recited in claim 30, wherein said additional herb mixture further comprises a predetermined amount of Cuttlefish Bone having a Chinese herb name of Hai Piao Xiao, provided for ceasing bleeding, restoring kidney deficiencies, and promoting healing of rashes and ulcers.

35. The skin condition relieving medication, as recited in claim 34, wherein said primary herb mixture comprises 20% by volume of said Forsythia, 20% volume of said Honeysuckle, 20% by volume of said Garden Angelica, 20% by volume of said Myrrh and 20% by volume of said Cuttlefish Bone.

36. The skin condition relieving medication, as recited in claim 25, wherein said cream is Vaseline.

37. The skin condition relieving medication, as recited in claim 22, wherein said medication further comprises one or more secondary herb, chosen from a secondary herb group of isatis leaf, isatis root, natural indigo, dandelion, viola, houttuynia, thlaspi, partulaca, smilax, dictamnus, belamcanda, sophora root, green chiretta, angelica root, Japaneses knottweed and dalbergia heartwood.
